(19) United States
(12) Patent Application Publication
(10) Pub. No.: US 2024/0043942 A1
(43) Pub. Date: Feb. 8, 2024

(54) An SSR Marker For Detection Of Broad Bean Varieties Resistant To Broad Bean Weevil And Application Thereof

(71) Applicant: Hainan University, Haikou City, Hainan Province (CN)

(72) Inventors: Aihua SHA, Jingzhou, Hubei Province (CN); Yinhua CHEN, Haikou City, Hainan Province (CN); Hu SUN, Jingzhou, Hubei Province (CN); Dan GONG, Jingzhou, Hubei Province (CN); Xiuxiu ZHU, Jingzhou, Hubei Province (CN); Haozhong JIANG, Jingzhou, Hubei Province (CN); Yantao XIANG, Jingzhou, Hubei Province (CN); Zhengxin WEI, Jingzhou, Hubei Province (CN)

(73) Assignee: Hainan University, Haikou City, Hainan Province (CN)

(21) Appl. No.: 17/624,413

(22) PCT Filed: Dec. 30, 2021
(86) PCT No.: PCT/CN2021/143335

§ 371 Date: Jan. 3, 2022

(30) Foreign Application Priority Data
Oct. 29, 2021 (CN) 202111277001.7

(51) Int. Cl. C12Q 1/6895

(52) U.S. Cl. CPC C12Q 1/6895; C12Q 2600/13; C12Q 2600/156

(57) ABSTRACT

The present invention provides an SSR marker for detecting broad bean varieties resistant to broad bean weevil and its application. The application of the SSR marker provided by the present invention to detect broad bean varieties resistant to broad bean weevil has the advantages for the detection method in reliability, simplicity and practicability. Therefore, the SSR marker provided by the present invention has important application prospects in the identification of broad bean germplasm resources and molecular marker-assisted breeding selection.

### TECHNICAL FIELD

The present invention relates to the field of biotechnology, in particular to an SSR marker for detection of broad bean varieties resistant to broad bean weevil and application thereof.

### BACKGROUND ARTS

Broad bean (Vicia faba L.), an annual herb, is rich in nutrition, containing 8 essential amino acids and 47% to 60% of carbohydrate. It is a crop for use as food, vegetables, feed, and green manure.

Broad bean weevil (Bruchus rufimanus) is a pest in the production and storage of broad bean. If no prevention and control measures are taken, the broad bean weevil can perforate 50% to 90% of the beans during the storage period, resulting in bitter taste, weight loss, and prone to mildew and deterioration, and reducing the germination rate by more than 20%. In recent years, the occurrence of broad bean weevil has become increasingly prominent, which has seriously affected the quality, yield and planting area of broad bean. The damage of broad bean weevil has become the most important factor affecting the development of broad bean industry.

At present, the main method for preventing and controlling the harm of broad bean weevil in production is aluminum phosphide fumigation, which not only increases the cost of broad bean production, but also easily leads to pesticide residues, causing environmental pollution and affecting the health of consumers. Therefore, cultivating broad bean varieties resistant to broad bean weevil has become the first choice to avoid the infestation of broad bean weevil. Traditional breeding methods are costly, time-consuming, and restrictive. The development of modern molecular biotechnology, especially the development of molecular marker technology, has greatly promoted the process of selection and breeding of new crop varieties. SSR markers have the advantages of good reproducibility, high polymorphism, codominant inheritance, and spread throughout the entire genome, making SSR markers a widely used molecular marker. The research and development of close-linked markers of broad bean weevil resistant genes, and the application of molecular marker-assisted selection breeding techniques to breed new varieties of broad bean weevil resistant broad beans are the most economical and environmentally friendly methods to prevent and control the harm of broad bean weevil, which is of great significance to reduce the harm of broad bean weevil and ensure the safe production of broad bean in China. However, because the broad bean genome is too large (about 14Gb), and the attention thereto is not very high, the genome sequence is not sequenced, and thus it is difficult to develop molecular markers. At present, there are few SSR markers in broad beans publicly published, which directly leads to the difficulty in breeding broad bean weevil resistant varieties through molecular marker-assisted selection.

### SUMMARY OF INVENTION

In view of this, the present invention provides a primer pair of SSR marker for assisting selection of broad bean weevil resistant broad beans and application thereof. The primer pair of SSR marker can be used for assisting selection of broad bean weevil resistant broad beans, and has remarkable selectivity for the traits of broad bean weevil resistance.

The inventors employ the PacBio third-generation full-length sequencing technology in combination with the RNA-seq method to sequence the whole genome of broad bean varieties. On this basis, the inventors search all Unigenes in the transcriptome by using the software MISA, seek for Unigenes containing SSR core motifs, then design primers based on these sequences and verify them through experimental methods, and finally obtain the nucleotide sequence containing a (TC)10 core motif in the present invention, as shown in SEQ ID NO: 7. According to the sequence of SEQ ID NO: 7, a primer pair of SSR for selecting broad beans resistant to broad bean weevil is developed, having a nucleotide sequence as shown in SEQ ID NO: 1 and SEQ. ID NO: 2. An experimental method to carry out SSR molecular marker selection is also developed.

One of the objectives of the present invention is to provide an SSR marker for detecting broad bean varieties resistant to broad bean weevil, wherein the SSR marker comprises an SSR core motif, a nucleotide sequence as shown in position 14-48 of SEQ ID NO: 7 which is located upstream of the core motif, and an nucleotide sequence as shown in position 114-242 of SEQ ID NO: 7 which is located downstream of the core motif; and the SSR core motif is a simple sequence repeat (SSR).

In some embodiments, the SSR core motif is (TC)n, where n≥5, for example, n is 10.

In some embodiments, the SSR marker comprises a nucleotide sequence as shown in position 14-242 of SEQ ID NO: 7.

Some embodiments provide an SSR marker for detecting broad bean varieties resistant to broad bean weevil, wherein the SSR marker comprises an SSR core motif, and the SSR marker comprises or has a nucleotide sequence as shown in SEQ ID NO: 7.

The second objective of the present invention is to provide a specific sequence containing the SSR core motif for the development of broad bean SSR markers. The SSR core motif is (TC)10, which is a sequence containing 10 consecutive TC repeats. The nucleotide sequence is shown in SEQ ID NO: 7:

5′-GTTATTGTCGTGGAGAAAGGAAAACCTCTCCCGCCACGAAAATTCT
ATTCTCTCTCTCTCTCTCTCTCTGTAGTAGCCATATACACAGCTAGCTT
CGCGTCACAATCTCACTCTCATCAACTCTTCAAACTAATGGCTGCAACT
CTTCAAACTCTGCGACTTCCTTTTCATCCATCTATCACACCCCTAAATT
CATATCCTTTCCCAACCTCTACCGTCCATTACTCCCCCAAATCAAGCAC
CTTCAAGGGTTCATCTGTCGCCACCCGCAACAAACCAGTACCTTCTTCC
AAAGCTTCCAATTCGCAGTACAGTCCCACTGTTACCGAAAATCTCGCTG
ACATTAGCATTTTCTCGGCCGCCGGTGAGCCCGTCATGTTCAAAGATCT
TTGGGACCAAGAACAGGAAAAGCCCTGATCTTTATCCATTTCTTATCTG
TTTTGGGCACAACCATTTTGACCTAAAATTGTGCTTTTTACTCTCTGGA
ATATTAATTTGAATTAGTTGAGTTAATGGGTTGATCTTAATTTGTGGTG
GGTGATTAGAGTTTATTCTTGCAGGGAATAGCTGTGGTTGCACTTTTAA
GGCACTTTGGATGTCCATGCTGCTGGGAATTGGCTTCAACTTTGAAAGA
ATCCAAATCAAGGTTTGATTCGGCTGGTGTGAAGTTAATTGCAGTGGGT
GTCGGTGCCCCCAATAAAGCCCGTCTCCTTGCCCAACGA-3′.

The third objective of the present invention is to provide a primer pair of SSR markers for assisting the identification of broad bean varieties resistant to broad bean weevil. The primer pair is used to amplify the SSR markers of broad bean varieties resistant to broad bean weevil, wherein:
the nucleotide sequence of the forward primer is AGAAAGGAAAACCTCTCCCG (SEQ ID NO: 1); and
the nucleotide sequence of the reverse primer is GTGCTTGATTTGGGGGAGTA (SEQ ID NO: 2).

The fourth object of the present invention is to provide a kit for identifying and/or selecting broad bean varieties resistant to broad bean weevil, comprising a forward primer with the sequence shown in SEQ ID NO: 1 and a reverse primer with the sequence shown in SEQ ID NO: 2.

The fifth object of the present invention is to provide application of said SSR marker in assisting identification of broad bean varieties resistant to broad bean weevil.

The sixth object of the present invention is to provide application of the primer pair for detecting SSR markers in assisting identification of varieties resistant to broad bean weevil.

The seventh objective of the present invention is to provide a method for assisting selection of broad bean varieties resistant to broad bean weevil, including the step of detecting an SSR marker of the broad bean varieties to be tested, wherein: the SSR marker comprises an SSR core motif, a nucleotide sequence as shown in positions 14-48 of SEQ ID NO: 7 which is located upstream of the core motif, and an nucleotide sequence as shown in positions 114-242 of SEQ ID NO: 7 which is located downstream of the core motif; and the SSR core motif is a simple sequence repeat (SSR).

In some embodiments, the SSR marker comprises a nucleotide sequence shown in position 14-242 of SEQ ID NO: 7.

In some embodiments, the SSR marker comprises a nucleotide sequence shown in SEQ ID NO: 7.

In some embodiments, the method includes the following steps:
(1) designing a primer pair according to the SSR marker, wherein the primer pair comprises a forward primer and a reverse primer, wherein the nucleotide sequence of the forward primer is AGAAAGGAAAACCTCTCCCG (SEQ ID NO: 1); and the nucleotide sequence of the reverse primer is GTGCTTGATTTGGGGGAGTA (SEQ ID NO: 2); and
(2) performing PCR amplification by using the genomic DNA of the broad bean variety to be tested as a template and the primer pair, and performing gel electrophoresis to identify whether the broad bean variety to be tested is a broad bean variety resistant to broad bean weevil.

In some embodiments, in step (2), according to the results of gel electrophoresis, if the band pattern of the PCR amplification product show as one of the four band patterns of A (three bands of 250 bp, 240 bp, and 235 bp), B (two bands of 250 bp and 235 bp), C (two bands of 248 bp and 240 bp) or D (two bands of 240 bp and 235 bp), the broad bean variety to be tested is identified as a broad bean variety resistant to broad bean weevil.

The eighth object of the present invention is to provide a method for assisting selection of broad bean varieties resistant to broad bean weevil, which includes the following steps:
Step 1. performing PCR amplification by using the genomic DNA of the broad bean variety to be tested as a template, and the primer pair shown in SEQ ID NO:1 and SEQ ID NO:2 to obtain PCR amplification product;
Step 2. performing gel electrophoresis for the PCR amplification product to detect the band pattern of the PCR product, wherein if the band pattern of the PCR amplification product show as one of the four band patterns of A (three bands of 250 bp, 240 bp, and 235 bp), B (two bands of 250 bp and 235 bp), C (two bands of 248 bp and 240 bp) or D (two bands of 240 bp and 235 bp), the broad bean variety to be tested is identified as a broad bean variety resistant to broad bean weevil.

Compared with the prior art, the present invention has the following beneficial effects.
(1) Stable labeling: this study identified 51 broad beans with different broad bean weevil resistances. Among the 8 broad bean varieties (lines) showing the A band pattern, 8 were materials with high resistance to broad bean weevil, the accuracy rate reaching 100%. Among the 15 broad bean varieties (strains) showing the B band pattern, 15 were materials with high resistance to broad bean weevil, the accuracy rate reaching 100%. Among the 10 broad bean varieties (strains) showing the C band pattern, 10 were materials with high resistance to broad bean weevil, and the accuracy rate reaching 100%. Among the 7 broad bean varieties (lines) showing the D band pattern, 6 were materials with high resistance to broad bean weevil, and the accuracy rate reaching more than 85%. It shows significant selectivity for traits of broad bean weevil resistance.
(2) Fast and accurate: With the method provided by the present invention, only the total DNA of broad bean needs to be extracted and amplified by PCR and then subjected to polyacrylamide gel electrophoresis, which can effectively identify the broad bean traits of broad bean weevil resistance and realize the first step of screening broad bean resistant to broad bean weevil. Therefore, this molecular marker has great application prospects in the future for assisting selection breeding of broad bean resistant to broad bean weevil.
(3) Lower requirements on the equipment used, and can be carried out in general molecular biology laboratories without complicated technical steps.

### BRIEF DESCRIPTION OF THE DRAWINGS

FIG. 1 is the results of polyacrylamide gel electrophoresis detection for 51 broad bean materials of the present invention, wherein lanes 1-51 represents the broad bean materials numbered 1-51; lanes 1-8 show A band pattern; lanes 9-23 show B band pattern; lanes 24-33 show C band pattern; lanes 34-39 and 49 show D band pattern; lanes 40 and 42 show E band pattern; lane 41 shows F band pattern; lanes 43-48, 50, and 51 show G band pattern.

FIG. 2 is the result of genomic DNA extraction of parts of broad beans of the present invention, wherein Figure A is the detection result of DNA extracted by the original CTAB method and Figure B is the detection result of DNA extracted by the improved CTAB method of the present invention.

FIG. 3 shows the screening of the primer and determination of primer annealing temperature of the present invention.

### DETAILED EMBODIMENTS

For better understanding of the technical content of the present invention, the present invention is further illustrated by the following examples.

Unless otherwise specified, the experimental methods used in the examples of the present invention are all conventional methods.

The materials, reagents, etc. used in the examples of the present invention can be obtained from commercial sources unless otherwise specified.

### Example 1-Acquisition of SSR Core Motif

The improved extraction method for broad bean genomic DNA includes the following steps:
(1) obtaining 80 mg of fresh young leaf tissue of broad bean, adding 700 μl DNA extract solution and 1% PVP (polyvinyl pyrrolidone), grinding thoroughly, and transferring the grinding liquid to the corresponding 1.5 mL centrifuge tube;
(2) putting the centrifuge tube in a 65° C. water bath for 30 minutes, taking the centrifuge tube out and shaking gently to mix every 5 minutes;
(3) after completion of the water bath, adding 500 μl of phenol/chloroform/isoamyl alcohol (v/v/v 25:24:1) in a fume hood, mixing it upside down, centrifuging at 12000 rpm for 10 minutes, transferring the supernatant to a new centrifuge tube, then adding the same amount of chloroform/isoamyl alcohol (v/v 24:1), and mixing upside down;
(4) centrifuging at 12000 rpm for 10 minutes, transferring 500 μl of supernatant to a new centrifuge tube, adding 2 times volume of pre-cooled absolute ethanol, mixing upside down, and then leaving it stand at −20° C. for 30 minutes; and
(5) centrifuging at 12000 rpm for 10 minutes, discarding the supernatant, adding 500 μl of 70% ethanol to wash, centrifuging at 12000 rpm for 10 minutes, discarding the supernatant, drying the edge of the centrifuge tube with clean paper, and absorbing the remaining liquid at the bottom of the centrifuge tube by using a micropipette, leaving it at room temperature to dry for 15 minutes, adding 40 μl of sterile water to dissolve the DNA precipitate, and performing electrophoresis using 1% agarose gel to detect the DNA quality after 1 day at room temperature.

The original method for extracting broad bean genomic DNA includes the following steps:
(1) obtaining 80 mg of fresh young leaf tissue of broad bean, adding 700 μl DNA extract solution, grinding thoroughly, and transferring the grinding liquid to the corresponding 1.5 mL centrifuge tube;
(2) water bathing at 65° C. for about 30 minutes, taking the centrifuge tube out and shaking gently to mix every 5 minutes;
(3) after completion of the water bath, adding 500 μl of chloroform/isoamyl alcohol (v/v 24:1) in a fume hood, mixing it upside down, centrifuging at 12000 rpm for 10 minutes, transferring 500 μl of the supernatant to a new centrifuge tube, then adding the same amount of isopropanol, mixing upside down, and then leaving it stand at room temperature for 20 minutes;
(4) centrifuging at 12000 rpm for 10 minutes, discarding the supernatant, and adding 500 μl of 70% ethanol to wash; and
(5) centrifuging at 12000 rpm for 10 minutes, discarding the supernatant, drying the edge of the centrifuge tube with clean paper, and absorbing the remaining liquid at the bottom of the centrifuge tube by using a micropipette, leaving it at room temperature to dry for 15 minutes, adding 40 μl of sterile water to dissolve the DNA precipitate, and performing electrophoresis using 1% agarose gel to detect the DNA quality after 1 day at room temperature.

As shown in FIG. 2, picture A is the detection result of DNA extracted by the original CTAB method, and picture B is the detection result of DNA extracted by the improved CTAB method of the present invention. It can be seen from the pictures that the improved CTAB method for DNA extraction of the present invention has higher extraction quality and greatly reduces DNA degradation.

### Example 2-SSR Core Motif Nucleotide Information

The whole genome of broad bean was sequenced by using PacBio third-generation full-length sequencing technology in combination with RNA-seq method. The sequence of SEQ ID NO: 7 was copied to MISA online software (http://pgrcipk-gatersleben.de/misa/), and analyzed with default parameters. It was found that the 48 bp-67 bp nucleotide sequence at the 5′end contains the SSR core motif of (TC)10. The nucleotide sequence is shown in SEQ ID NO: 7:

5′-GTTATTGTCGTGGAGAAAGGAAAACCTCTCCCGCCACGAAAATTCT
ATTCTCTCTCTCTCTCTCTCTCTGTAGTAGCCATATACACAGCTAGCTT
CGCGTCACAATCTCACTCTCATCAACTCTTCAAACTAATGGCTGCAACT
CTTCAAACTCTGCGACTTCCTTTTCATCCATCTATCACACCCCTAAATT
CATATCCTTTCCCAACCTCTACCGTCCATTACTCCCCCAAATCAAGCAC
CTTCAAGGGTTCATCTGTCGCCACCCGCAACAAACCAGTACCTTCTTCC
AAAGCTTCCAATTCGCAGTACAGTCCCACTGTTACCGAAAATCTCGCTG
ACATTAGCATTTTCTCGGCCGCCGGTGAGCCCGTCATGTTCAAAGATCT
TTGGGACCAAGAACAGGAAAAGCCCTGATCTTTATCCATTTCTTATCTG
TTTTGGGCACAACCATTTTGACCTAAAATTGTGCTTTTTACTCTCTGGA
ATATTAATTTGAATTAGTTGAGTTAATGGGTTGATCTTAATTTGTGGTG
GGTGATTAGAGTTTATTCTTGCAGGGAATAGCTGTGGTTGCACTTTTAA
GGCACTTTGGATGTCCATGCTGCTGGGAATTGGCTTCAACTTTGAAAGA
ATCCAAATCAAGGTTTGATTCGGCTGGTGTGAAGTTAATTGCAGTGGGT
GTCGGTGCCCCCAATAAAGCCCGTCTCCTTGCCCAACGA-3′.

### Example 3-Assisted Identification of Broad Bean Varieties Resistant to Broad Bean Weevil with SSR Markers

Step 1. Primer design: According to the nucleotide sequence SEQ ID NO: 7 of the specific broad bean genome containing the SSR core motif (TC)10, 3 pairs of SSR primers were designed with Primer 3 software. The sequences are:

SEQ ID NO: 1:
AGAAAGGAAAACCTCTCCCG;
SEQ ID NO: 2:
GTGCTTGATTTGGGGGAGTA. (The first pair of primers)
SEQ ID NO: 3:
AGAAAGGAAAACCTCTCCCG;
SEQ ID NO: 4:
GAGTGAGATTGTGACGCGAA. (The second pair of primers)
SEQ ID NO: 5:
AGAAAGGAAAACCTCTCCCG;
SEQ ID NO: 6:
AAGGTACTGGTTTGTTGCGG. (The third pair of primers)

Step 2. PCR amplification: the PCR conditions exploration.

The initial annealing temperature of the primer was obtained by decreasing the Tm value by 3° C. according to the primer synthesis sheet. PCR amplification was carried out by using genomic DNA of parts of the broad beans to be tested as a template, and using the above 3 pairs of primers.

The total PCR reaction system is 15 μL, including each 0.6 μL of Primer F and Primer R primers, 1.5 μL of template DNA, 9.4 μL of ddH₂O, 0.3 μL of dNTPs, 1.5 μL of 10×buffer, 0.9 μL of Mg²⁺, and 0.2 μL of thermostable DNA polymerase (Taq enzyme).

PCR amplification was performed on the TGreat Gradient Thermal Cycler (96 Well) OSE-GP-01 PCR instrument, with the lid temperature being controlled at 105° C., and the pre-denaturation at 95° C. for 5 min; then 35 cycles of pre-denaturation (95° C., 30 s)), annealing (the temperature varies with the primer for 30 s), extension (72° C., 45 s), then continuing to extend at 72° C. for 10 minutes, and finally, slowly cooling to 4° C.

Step 3. Gel detection

½ volume of denaturant (10 mL of 5×TBE, 90 mL of formamide, 0.05 g of bromophenol blue, and 0.05 g of xylene cyanol FF) was added to the amplification product of step 2 and denatured at a constant temperature of 95° C. for 5 minutes. 4 μL was used for a 6% polyacrylamide gel electrophoresis to separate, and silver-staining detection is performed. The band pattern was observed and analyzed.

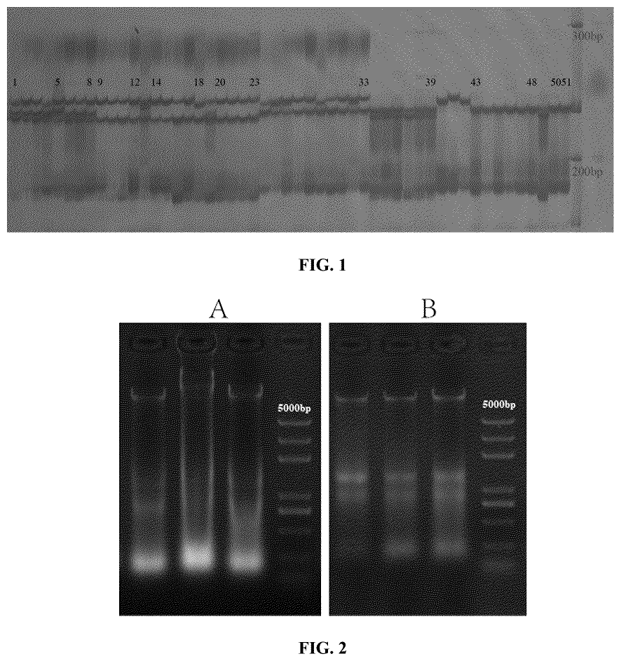

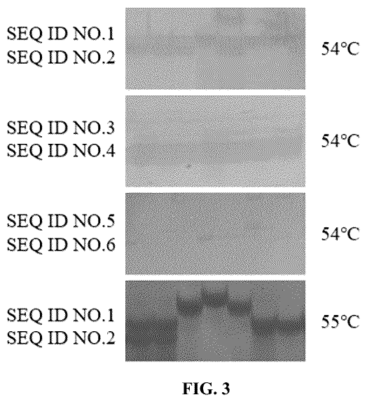

As shown in FIG. 3, when the three pairs of primers were initially screened at an annealing temperature of 54° C., only the first pair of primers amplified specific bands, the second pair of primers amplified non-specifically, and the third pair of primers had serious dispersion, so the latter two pairs of primers they were discarded. At the annealing temperature of 55° C., the primers of SEQ ID NOs: 1 and 2 have a good amplification effect. Therefore, the first pair of primers was selected to identify 51 broad bean varieties, and the results were shown in FIG. 1 and Table 1.

TABLE 1
The broad bean weevil resistance and SSR band pattern
results of 51 broad bean varieties (lines)

| Serial No. | Name of variety | resistance | SSR band pattern |
|---|---|---|---|
| 1 | H0000152 | HR | A |
| 2 | H0000160 | HR | A |
| 3 | H0001419 | HR | A |
| 4 | H0001426 | HR | A |
| 5 | H0001516 | HR | A |
| 6 | H0001522 | HR | A |
| 7 | H0003827 | HR | A |
| 8 | H050 | HR | A |
| 9 | H0000153 | HR | B |
| 10 | H0000158 | HR | B |
| 11 | H0001454 | HR | B |
| 12 | H0001484 | HR | B |
| 13 | H0001485 | HR | B |
| 14 | H0001491 | HR | B |
| 15 | H0001530 | HR | B |
| 16 | H0003833 | HR | B |
| 17 | H0003879 | HR | B |
| 18 | H003 | HR | B |
| 19 | H011 | HR | B |
| 20 | H044 | HR | B |
| 21 | H049 | HR | B |
| 22 | H071 | HR | B |
| 23 | P420684008 | HR | B |
| 24 | H0001401 | HR | C |
| 25 | H0001427 | HR | C |
| 26 | H0001458 | HR | C |
| 27 | H0001489 | HR | C |
| 28 | H0003874 | HR | C |
| 29 | H001-1 | HR | C |
| 30 | H004 | HR | C |
| 31 | H012 | HR | C |
| 32 | H037 | HR | C |
| 33 | H111 | HR | C |
| 34 | H0001504 | HR | D |
| 35 | H0003838 | HR | D |
| 36 | Id0027 | HR | D |
| 37 | H060 | HR | D |
| 38 | 2015422285 | HR | D |
| 39 | 2015422397 | HR | D |
| 40 | H0003859 | HS | E |
| 41 | H115 | HS | F |
| 42 | Y04-008 | HS | E |
| 43 | Y04-210-1-1 | HS | G |
| 44 | Y04-215-1-1 | HS | G |
| 45 | Y05-131-4 | HS | G |
| 46 | Y05-131-11-1-1 | HS | G |
| 47 | Y10-613 | HS | G |
| 48 | Jing 04-212-1-4 | HS | G |
| 49 | Id0018 | S | D |
| 50 | Y01-102-1-91 | S | G |
| 51 | Y01-102-2 | S | G |

It can be seen from FIG. 1 that among the 33 broad bean varieties (lines) showing the three band patterns of A, B, and C, 33 were broad bean materials with high resistance to broad bean weevil, and the accuracy rate reached 100%. Among the 7 broad bean varieties (lines) showing D band pattern, 6 were broad bean materials with high resistance to broad bean weevil, and the accuracy rate reached more than 85.7%. It shows significant selectivity for traits of broad bean weevil resistance.

The above mentioned are only preferred embodiments of the present invention and are not intended to limit the present invention. Any modification, equivalent replacement, improvement etc. made within the spirit and principle of the present invention shall be included in the scope of protection of the present invention.

1. An SSR marker for detecting broad bean varieties resistant to broad bean weevil, wherein the SSR marker is a specific broad bean genome sequence containing an SSR core motif, and the sequence is shown in SEQ ID NO: 7.

2. The SSR marker according to claim 1, wherein a method for obtaining the specific broad bean genome sequence containing the SSR core motif comprises: sequencing whole genome of the broad bean varieties by using PacBio third-generation full-length sequencing technology in combination with RNA-seq method and searching for Unigenes in the transcriptome by software MISA.

3. The SSR marker according to claim 1, wherein the specific broad bean genome sequence containing the SSR core motif is a nucleotide sequence containing 10 consecutive TC repeats.

4. The SSR marker according to claim 1, wherein a primer pair is designed according to the SSR core motif, and the sequence of the primer pair comprises:
a forward primer, the nucleotide sequence of which is shown in SEQ ID NO:1: 5′-AGAAAGGAAAACCTCTCCCG-3′; and
a reverse primer, the nucleotide sequence of which is shown in SEQ ID NO: 2: 5′-GTGCTTGATTTGGGGGAGTA-3′.

5. Application of the SSR marker according to claim 1 for assisting identification of broad bean varieties resistant to broad bean weevil.

6. The application according to claim 5, wherein it comprises the following steps:
(1) designing a primer pair according to the SSR marker, wherein the primer pair comprises a forward primer and a reverse primer, wherein the nucleotide sequence of the forward primer is AGAAAGGAAAACCTCTCCCG (SEQ ID NO:1) and the nucleotide sequence of the reverse primer is GTGCTTGATTTGGGGGAGTA (SEQ ID NO: 2); and
(2) performing PCR amplification by using the genomic DNA of the broad bean variety to be tested as a template and the primer pair, and performing gel electrophoresis to identify whether the broad bean variety to be tested is a broad bean variety resistant to broad bean weevil.

7. The application according to claim 6, wherein the PCR amplification reaction system comprises: each 0.6 μL of forward primers and reverse primers, 1.5 μL of template DNA, 9.4 μL of ddH₂O, 0.3 μL of dNTPs, 1.5 μL of 10×buffer, 0.9 μL of Mg 2+, and 0.2 μL of thermostable DNA polymerase.

8. The application according to claim 6, wherein the PCR amplification program includes: a lid temperature of 105° C., a constant temperature of 95° C. for pre-denaturation for 5 minutes, and 35 cycles, the conditions of each cycle are: denaturation at 95° C. for 30 s, annealing at 55° C. for 30 s, extension at 72° C. for 45 s, extension at 72° C. for 10 min after the cycle, and finally cooling to 4° C.

9. The application according to claim 6, wherein when the gel electrophoresis shows A band pattern, B band pattern, C band pattern or D band pattern, it is determined that the broad bean variety is a broad bean variety resistant to broad bean weevil; and wherein the A band pattern results in three bands of 250 bp, 240 bp and 235 bp; the B band pattern results in two bands of 250 bp and 235 bp; the band pattern results in two bands of 248 bp and 240 bp; and the D band pattern results in two bands of 240 bp and 235 bp.

10. A method for assisting selection of broad bean varieties resistant to broad bean weevil, including the step of detecting an SSR marker of broad bean varieties to be tested, wherein:
the SSR marker comprises an SSR core motif, a nucleotide sequence as shown in position 14-48 of SEQ ID NO: 7 which is located upstream of the core motif, and an nucleotide sequence as shown in position 114-242 of SEQ ID NO: 7 which is located downstream of the core motif; and the SSR core motif is a simple sequence repeat (SSR).

11. The method according to claim 10, wherein the SSR marker comprises a nucleotide sequence as shown in position 14-242 of SEQ ID NO: 7.

12. The method of claim 10, wherein the SSR marker comprises a nucleotide sequence shown in SEQ ID NO.:7.

13. The method according to claim 10, comprising the following steps:
(1) designing a primer pair according to the SSR marker, wherein the primer pair comprises a forward primer and a reverse primer, wherein the nucleotide sequence of the forward primer is AGAAAGGAAAACCTCTCCCG (SEQ ID NO:1); the nucleotide sequence of the reverse primer is GTGCTTGATTTGGGGGAGTA (SEQ ID NO: 2); and
(2) performing PCR amplification by using the genomic DNA of the broad bean variety to be tested as a template and using the primer pair, and performing gel electrophoresis to identify whether the broad bean variety to be tested is a broad bean variety resistant to broad bean weevil.

14. The method according to claim 13, wherein in step (2), when the gel electrophoresis results show A band pattern, B band pattern, C band pattern or D band pattern, it is determined that the corresponding broad bean variety to be detected is a broad bean variety resistant to broad bean weevil, wherein the A band pattern results in three bands of 250 bp, 240 bp and 235 bp; the B band pattern results in two bands of 250 bp and 235 bp; the band pattern results in two bands of 248 bp and 240 bp; and the D band pattern results in two bands of 240 bp and 235 bp.

15. A primer pair for detecting broad bean varieties resistant to broad bean weevil, the primer pair includes a forward primer and a reverse primer, wherein:
the nucleotide sequence of the forward primer is AGAAAGGAAAACCTCTCCCG (SEQ ID NO: 1); and the nucleotide sequence of the reverse primer is GTGCTTGATTTGGGGGAGTA (SEQ ID NO: 2).

16. A method for identifying broad bean varieties resistant to broad bean weevil, comprising the step of detecting an SSR marker in broad bean varieties to be detected by using the primer pair of claim 15.

17. Application of the SSR marker according to claim 3 for assisting identification of broad bean varieties resistant to broad bean weevil.

18. The application according to claim 17, wherein it comprises the following steps:
(1) designing a primer pair according to the SSR marker, wherein the primer pair comprises a forward primer and a reverse primer, wherein the nucleotide sequence of the forward primer is AGAAAGGAAAACCTCTCCCG (SEQ ID NO:1) and the nucleotide sequence of the reverse primer is GTGCTTGATTTGGGGGAGTA (SEQ ID NO: 2); and
(2) performing PCR amplification by using the genomic DNA of the broad bean variety to be tested as a template and the primer pair, and performing gel electrophoresis to identify whether the broad bean variety to be tested is a broad bean variety resistant to broad bean weevil.

19. The application according to claim 18, wherein the PCR amplification reaction system comprises: each 0.6 μL of forward primers and reverse primers, 1.5 μL of template DNA, 9.4 μL of ddH₂O, 0.3 μL of dNTPs, 1.5 μL of 10×buffer, 0.9 μL of Mg 2+, and 0.2 μL of thermostable DNA polymerase.

20. The application according to claim 18, wherein the PCR amplification program includes: a lid temperature of 105° C., a constant temperature of 95° C. for pre-denaturation for 5 minutes, and 35 cycles, the conditions of each cycle are: denaturation at 95° C. for 30 s, annealing at 55° C. for 30 s, extension at 72° C. for 45 s, extension at 72° C. for 10 min after the cycle, and finally cooling to 4° C.


SEQUENCE LISTING (8 sequences)

SEQ ID NO: 1 - protein, 503 aa, Vigna angularis
MAQVQVQPQNAMPGPNGAAAAAGGNQFVTTSLYVGDLDPNVTDSQLYDLFSQLGQVVSVRVCRDLTSRRSLGYGYVNYSNPQDAARALDVLNFTPLNNKPIRIMYSHRDPCIRKSGAGNIFIKNLDRAIDHKALHDTFSTFGNILSCKVATDSSGQSKGYGFVQFDNEESAQKAIEKLNGMLLNDKQVYVGPFLRKQERETAIDKAKFNNVFVKNLADSTSDDELKTIFGEFGTITSAVVMRDGDGKSKCFGFVNFENADDAARAVEALNGKKFDDKEWYVGKAQKKSERENELKQRFEQSMKEAADKYQGANLYVKNLDDSISDDKLKELFSPFGTITSCKVMRDPNGVSRGSGFVAFSTPEEASRALSEMNGKMVVSKPLYVTLAQRKEDRRARLQAQFAQMRPVGMPPSVGPRVPMYPPGGPGIGQQIFYGQGPPAIIPSQAGFGYQQQLVPGMRPGAAPVPNFFVPMVQQGQQGQRPGGRRAVQQSQQPVPMMPQQMLP

SEQ ID NO: 2 - DNA, 1511 bp, Artificial Sequence
  cDNA
atggctcaggttcaggttcagcctcagaatgcgatgcccggtcccaacggtgctgctgctgctgctgggggaaaccagttcgttacgacatcgctttacgtcggagatctcgaccccaacgtcacggactcacagctttatgacctgttcagtcaattgggccaagttgtgtctgttagggtttgcagggacttgaccagccgaagatcgctcggttacggctatgtcaactatagcaacccccaagatgctgccagagcattagatgttctgaatttcactcctctcaacaacaagcccatccgaattatgtattcacatcgtgatccctgtatccggaaaagtggggcaggaaatatttttatcaagaatttggatagggcaattgaccacaaggcattacatgataccttctctacatttgggaatatcctttcatgcaaggtagcaacggattcatctgggcaatcaaaaggatatggttttgttcagtttgataatgaggaatctgcccaaaaagccatagagaagctgaatggtatgctgttgaatgataagcaagtgtatgtgggacccttccttcgcaagcaagagagagagactgctattgacaaggcaaaattcaataatgtttttgtaaagaatctagcagattcgactagtgatgatgaattgaagacaatttttggtgaatttggaactattactagtgctgtagtgatgagggatggagatgggaaatcaaagtgctttgggtttgtgaattttgagaatgctgatgatgctgctagggctgttgaggctctcaatggcaaaaaatttgatgataaggaatggtacgttggaaaagctcagaagaaatctgaaagggagaatgaattgaaacaacgatttgagcagagcatgaaagaagctgctgataaatatcaaggggcaaacttgtatgtcaaaaatttggatgatagcattagtgatgataaacttaaggagctgttctccccttttggtaccatcacctcttgcaaggttatgagggacccaaatggcgttagtcgtggatctggatttgttgcattctcaactcctgaggaggcatctagagcactctctgagatgaatgggaaaatggtggtaagtaaacctctgtatgtgactctagcccaaaggaaagaagatagaagagctagactgcaggctcagtttgctcaaatgcgacctgttggaatgccaccatctgttggtcctcgtgtgccaatgtatcctccaggtggtccaggtattggtcaacaaatattttatggccaaggccctcctgctatcattccttcccaggccggatttggttaccaacaacaacttgtgcctggtatgaggccaggtgcagctcctgtgccaaatttctttgtgccaatggttcagcagggacaacagggccagcgccctggtggaaggcgtgcagtccagcagtcccagcagccagttccaatgatgccacagcagatgcttcctag

SEQ ID NO: 3 - DNA, 36 bp, Artificial Sequence
  forward primer
cgacgacaagaccctatggctcaggttcaggttcag

SEQ ID NO: 4 - DNA, 36 bp, Artificial Sequence
  reverse primer
gaggagaagagcccctaggaagcatctgctgtggca

SEQ ID NO: 5 - DNA, 18 bp, Artificial Sequence
  forward primer
cgctcggttacggctatg

SEQ ID NO: 6 - DNA, 18 bp, Artificial Sequence
  reverse primer
gcttgcgaaggaagggtc

SEQ ID NO: 7 - DNA, 19 bp, Artificial Sequence
  forward primer
ccctcccacatgctattct

SEQ ID NO: 8 - DNA, 19 bp, Artificial Sequence
  reverse primer
agagcctccaatccagaca